(19) United States
(12) Patent Application Publication
(10) Pub. No.: US 2016/0331811 A1
(43) Pub. Date: Nov. 17, 2016

(54) NON-AGGLOMERATING BIOCONJUGATES OF AMYLIN-MIMETIC COMPOUNDS AND POLYETHYLENEGLYCOL

(71) Applicant: UNIVERSIDADE FEDERAL DO RIO DE JANEIRO, Rio de Janeiro RJ (BR)

(72) Inventors: Luis Mauricio TRAMBAIOLI DA ROCHA E LIMA, Rio de Janeiro, RJ (BR); Luiz Henrique GUERREIRO ROSADO, Seropédica, RJ (BR); Mariana Fernandes de AVILA NETTO GUTERRES, Rio de Janeiro, RJ (BR); Bruno MELO VIEIRA GONÇALVES FERREIRA, Rio de Janeiro, RJ (BR)

(21) Appl. No.: 14/898,237

(22) PCT Filed: Jun. 13, 2014
(86) PCT No.: PCT/BR2014/000199

§ 371 Date: Dec. 14, 2015

(30) Foreign Application Priority Data
Jun. 14, 2013 (BR) BR 102013017626-5

(51) Int. Cl. A61K 38/22; A61K 45/06; A61K 38/28; A61K 47/48

(52) U.S. Cl. CPC A61K 38/22; A61K 47/48215; A61K 45/06; A61K 38/28

(57) ABSTRACT

The present invention generally concerns new non-agglomerating bioconjugates of amylin-mimetic compounds with polyethylene glycol, and their use mainly in the treatment of diseases associated with extracellular amyloid deposition or accumulation that contributes to the dysfunction or failure of systemic organs such as the pancreas.

The present invention generally concerns new non-agglomerating bioconjugates of amylin-mimetic compounds and polyethylene glycol, their use mainly in the treatment of diseases associated with deposition or accumulation of extracellular amyloid, which contributes to the dysfunction or failure of systemic organs such as the pancreas.

A particular embodiment of the invention, without excluding any other, concerns the bioconjugates of natural or synthetic human amylin and polyethylene glycol, and the use thereof in the treatment of diabetes, and the toxic effects caused by build-up of amylin oligomers on the surface of pancreatic beta-cells membrane. Said build-up contributes to with the pathogenesis of diabetes. Preserving those cells, already deteriorated in diabetic patients, is therefore a benefit sought for in the art.

### BACKGROUND

Amylin, an amyloid polypeptide of the Langerhans islets, identified with CAS RN: 106602-62-4, is a hormone of the pancreatic beta-cells, co-secreted with insulin in response to feeding, and complements the effects of insulin on the control of postprandial glucose. It is a 37-aminoacid polypeptide, co-secreted with insulin in a proportion insulin:amylin of 100:1.

The beneficial activities of amylin in diabetes and obesity are known: increase in satiety leading to reduced ingestion of food and consequent body weight reduction; slower gastric emptying, improved glucose metabolism profiles with postprandial peaks and reduction of glucagon levels in diabetic patients.

Type 1 diabetes patients have practically no natural amylin production, while patients with long-standing type 2 diabetes have lower levels than healthy individuals.

Because of its physicochemical properties, the use of structurally unmodified amylin as a pharmaceutical active is not possible. Its limited solubility in aqueous solution led to the development of a more soluble amylin-mimetic compound, the pramlintide, in which three of 37 aminoacids of murin amylin are replaced by proline. There is a corresponding commercial product of pramlintide named Symlin®.

Although the solubility of pramlintide is greater than that of human amylin, pramlintide is more similar to murine amylin than human, and does not have good stability in neutral pH, so the product Symlin® is provided as an acid solution. Murine amylin shows reduced propensity of amyloid agglomeration compared to human amylin, suggesting a general propensity for amylin and amylin-mimetic substances. This product has to be administered as subcutaneous injections right before the meals, aiming to increase postprandial amylin levels. Because of its short half-life from 10 to 15 min, such injections of pramlintide increase the concentration of amylin in the blood stream as a series of peaks, not able to restore the basal levels of amylin during fasting. In the scientific literature, pramlintide has been associated with increased risk of severe insulin-induced hypoglycemia, and other adverse effects such as nausea, vomiting, anorexia and fatigue.

The technical literature does not appear to have focused on a solution for the agglomerating tendency of human amylin, or amylin deposition and its harmful effects on pancreatic beta-cell membranes; and no solution is available.

Without being bound by theory, there are indications that cholesterol in the plasma membrane of beta-cells is the key factor in regulating the so-called polymerization of amylin, and its deposition upon said membranes. Absence or low level of cholesterol in the plasma membrane—which is an ordinary aspect in diabetes—inhibits the internalization of amylin oligomers, and favors the extracellular accumulation, potentiating amylin cytotoxicity in that state, said toxicity resulting apoptosis or destruction of beta-cells. On the other hand, the cellular internalization of monomeric amylin is independent of the cholesterol content of the plasma membrane.

Briefly, a series of studies correlates amylin polymerization and deposition—with corresponding toxicity—on the surface of the plasma membrane of beta-cells with absence or lesser presence of cholesterol and/or with increased severity of type 2 diabetes mellitus.

Up to the present there are no medicaments or pharmaceutical actives known for the control of glycemia that also inhibit the aggregation of human amylin or amylin-mimetic compounds.

The present invention generally aims at the conjugation of human amylin and polyethylene glycol, and the use thereof towards the inhibition of the extracellular aggregation of amylin, without inducing the toxicity related to the formation of amylin oligomers and clusters.

The text that follows is to be understood with the following aspects: (1) mention of bioconjugates of amylin with polyethylene glycol also encompasses bioconjugates of amylin-mimetic compounds with polyethylene glycol; (2) the expression “amylin-mimetic compounds” also includes, for ease of expression, human amylin, natural or synthetic, (3) mention of diabetes also encompasses other diseases and dysfunctions directly or indirectly related to the agglomeration/deposition of amylin-mimetic compounds.

The use of the bioconjugates of the invention, particularly bioconjugates of human amylin, shows various benefits, particularly higher stability of the diabetic organism, that are highly recommended by physicians as desirable characteristics for medicaments of this nature:
increase of the plasma half-life, compared with amylin and amylinomimetic substances such as pramlintide, therefore allowing longer permanence in the blood stream;
lower doses or fewer injections to mimic the effect of natural amylin;
good control of glycemia;
reduction of nausea;
high satiety concerned with feeding;
reduced speed of gastric emptying;
higher solubility than human amylin;
reduction of glucagon levels, therefore improving glucose control.

### DESCRIPTION OF DRAWINGS

FIG. 1—chromatogram for monopegylated amylin obtained by the reaction of human amylin and mPEG;

FIG. 2—Mass spectrometry showing characterization of monopegylated human amylin;

FIGS. 3 and 4—assays for RAMP2 and RAMP3 coreceptor binding interaction for free and purified monopegylated human amylin;

FIG. 5—assay for amyloid aggregates;

FIG. 6—plasma stability comparison between free amylin and monopegylated human amylin

### DESCRIPTION OF THE INVENTION

The invention generally concerns chemical entities that aim to avoid the typical toxicity caused by human amylin and amylin-mimetic compounds cause, by decreasing or avoiding agglomeration (also mentioned in the literature as polymerization), deposition and fibrillation upon the pancreatic beta-cells and, in consequence, avoiding harmful effects that apoptosis or destruction of said pancreatic beta-cells cause to the human organism.

In a first aspect, the invention concerns new non-agglomerating bioconjugates of amylin or amylin-mimetic compounds and polyethylene glycol, characterized by the fact that said bioconjugate contains at least one polyethylene glycol unit (therefore monoconjugate or polyconjugate compounds may be obtained), covalently bonded to the two nitrogen atoms originated from the alpha and epsilon amine moieties (lateral chain) of the lysine 1 residue of the amylin polypeptidic chain.

It is therefore an object of the invention non-agglomerating bioconjugates of amylin-mimetic compounds of formula I

(R1-COX)m—R2

where
R1 represents methoxypolyethylene glycol (mPEG) moiety and functional spacers with various average molar masses,
R2 represents amylin or amylin-mimetic compounds,
X represents NH or O,
m represents the number of units of the mPEG polymer (R1) conjugated to amylin-mimetic compounds (R2) obtained from the conjugation of mPEG-succinimidyl with amylin-mimetic compounds, including amylin-mimetics devoid of Lys1 (or: des-Lys1), through an amide or ester bond by reaction of primary amine or hydroxyl functional moieties;
   and compounds of formula II

(R1-X)m-R2

where
R1 represents methoxypolyethylene glycol (mPEG) moiety and functional spacers with various average molar masses,
R2 represents amylin or amylin-mimetic compounds,
X represents NH or O,
m represents the number of units of the mPEG polymer (R1) conjugated to amylin-mimetic compounds (R2) obtained from the conjugation of mPEG-aldehyde with amylin-mimetic compounds.

Mention of amylin-mimetic compounds in the bioconjugates of the invention encompasses active derivatives of said amylin-mimetic compounds such as salts, isomers, hydrates, solvates, prodrugs, metabolites, polymorphs and isosteres.

It is important to remember that mention of amylin-mimetic compounds, in this text, encompasses human amylin itself, either natural, synthetic or bio-semi-synthetic.

In another aspect, the present invention concerns the use of the new non-agglomerating bioconjugates of amylin-mimetic compounds with polyethylene glycol, in the prevention or treatment of diseases or problems, caused or favored by the amyloid deposition or accumulation that leads to dysfunction or failure of systemic organs (i.e. organs or tissues external to the central nervous system), such as hyperglycemia, diabetes, low tolerance to glucose or deficient glucose metabolism, obesity, metabolic syndrome and feeding disorders, and indirectly to problems and vascular diseases resulting from increase in blood pressure, such as atherosclerosis, myocardial infarction, stroke, coronary heart disease, cardiac diseases in general, Alzheimer disease.

In another aspect, the invention concerns the use of the new non-agglomerating bioconjugates of amylin-mimetic compounds with polyethylene glycol for the preparation of low toxicity products, medicaments, compositions and associations, useful in the prevention or treatment of diseases caused or favored by amyloid deposition or accumulation which leads to dysfunction or failure of systemic organs.

Still in another aspect, the invention concerns low toxicity pharmaceutical compositions comprising a therapeutically effective amount of one or more of the new non-agglomerating bioconjugates of amylin-mimetic compounds with polyethylene glycol and one or more pharmaceutically acceptable excipients. Such compositions are adequate for all variety of administration forms such as oral, enteral, parenteral, lingual, sublingual, nasal, dermal, epidermal, transdermal, mucosal, vaginal, rectal, ocular, etc.

The compositions of the invention present themselves in any necessary or adequate dosage forms, such as solutions, suspensions, emulsions, microemulsions, foams, pastes, creams, tablets, capsules (hard or soft, suppositories), bolus, gels, powders, aerosols, sprays, etc.

The pharmaceutically acceptable excipients employed in the compositions of the inventions are known to the person skilled in the art, such as the ones described, for instance, in “Remington's Pharmaceutical Sciences”, 15th edition, Mack Publishing Co., New Jersey (1991). As known, specific excipients are chosen according to the desired administration route, within the practice of the pharmacological area.

The pharmaceutical compositions of the invention may additionally comprise one or more active principles, distinct from human amylin, such as (without excluding any other) insulin, ions (such as zinc and sodium), antidiabetics, antibiotics, anti-hypertensives, antiretrovirals, etc. Such compositions may be of immediate, retarded or slow release, also including the possibility that the administration of the new bioconjugate of human amylin be concomitant or sequential to other active principles.

Still another aspect of the invention concerns the use of non-agglomerating bioconjugates of human amylin and polyethyleneglycol as an adjuvant in the prevention or treatment of diseases caused or favored by amyloid deposition or accumulation that leads to dysfunction or failure of systemic organs.

Still another aspect of the invention concerns a medicament characterized by the fact that it comprises a therapeutically effective amount of one or more bioconjugates of human amylin and polyethylene glycol.

Still another aspect of the invention concerns bioconjugates of human amylin and polyethylene glycol, as well as products, medicaments, compositions and associations that comprise them, characterized for the use in medical therapy.

Still another aspect of the invention concerns a method or treatment or prevention of diseases caused or favored by amyloid deposition or accumulation, characterized by comprising the administration to a patient of a therapeutically effective amount of one or more bioconjugates of amylin-mimetic compounds, particularly human amylin, and polyethylene glycol.

### EXAMPLES

The examples that follow concern particular embodiments of the present invention and are not intended to, in any way, limit the scope of the attached claims.

### 1. Preparation of a Conjugate According to the Invention

Reaction for 2 h at 25° C. of a 5 mg/mL human amylin solution in the presence of 10 mM PBS (phosphate buffer solution) pH 7.4, and a molar excess of 5 mPEG/1 human amylin.

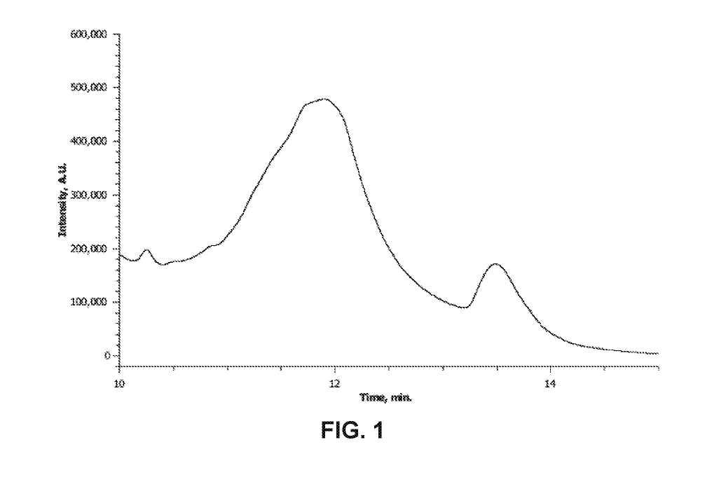

The reaction is quenched by the addition of an equal amount of 30% acetonitrile/0.1% trifluoroacetic acid (in water) and then chromatographed in a C18 Kromasil reversed phase column at 4 mL/min, detector set at 220 nm. (Kromasil is a product line commercialized by Separation Products group, a department of AkzoNobel company, Sweden). This purification process is shown in FIG. 1. The chromatogram shows a peak of monoPEGylated amylin at 12 minutes.

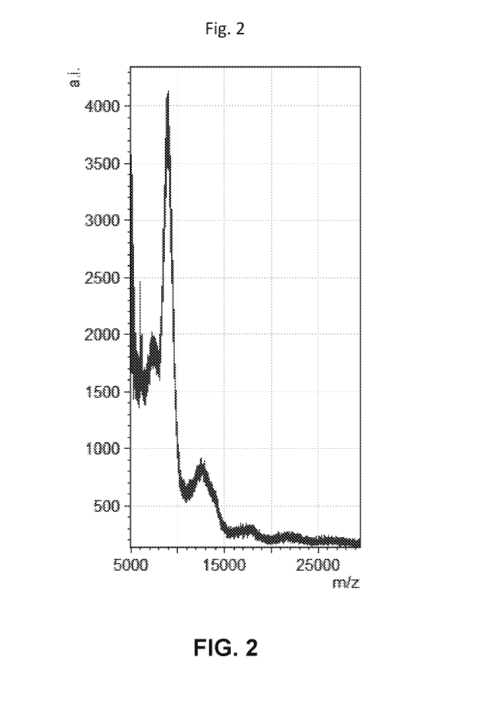

Matrix-assisted laser desorption and ionization-time-of flight mass spectrometry (MALDI-TOF-MS) was performed to characterized monoPEGylated human amylin. FIG. 2 shows the peak of monoPEGylated human amylin at 9 Kda.

### 2. Receptor Binding Assay of Human Amylin and MonoPEGylated Human Amylin

Purified monoPEGylated human amylin was assayed for RAMP2 and RAMP3 coreceptor binding interaction.

RAMP (receptor activity modifying protein) was labeled with fluorescein isothiocianate for 1 h at 4° C. in PBS (fetal bovine serum) pH 7.4, and purified by size exclusion chromatography (SEC) in Sepharose G25 (agarose-based chromatography media, provided by the US company GE HealthCare) using the same buffer. Labelling was confirmed by UV absorbance measurements at A280 and A490, allowing estimation of coupling efficiency, relying on about 0.5 fluorescein/RAMP molecule. Binding was performed by measuring the fluorescence anisotropy of RAMP-FITC (fluorescein isothiocyanate) as a function of free murine amylin, human amylin-PEGylated and by using the unrelated protein hen egg white lisozyme (HEWL) as a control for non-specific binding.

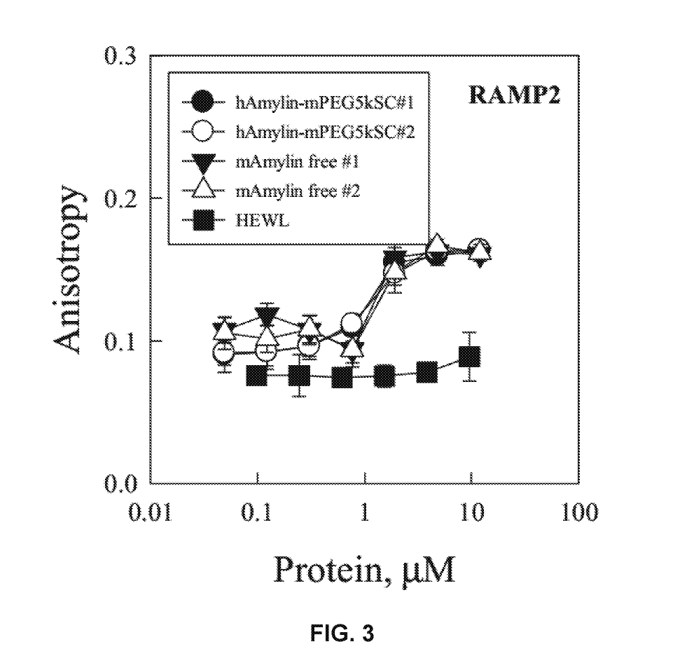

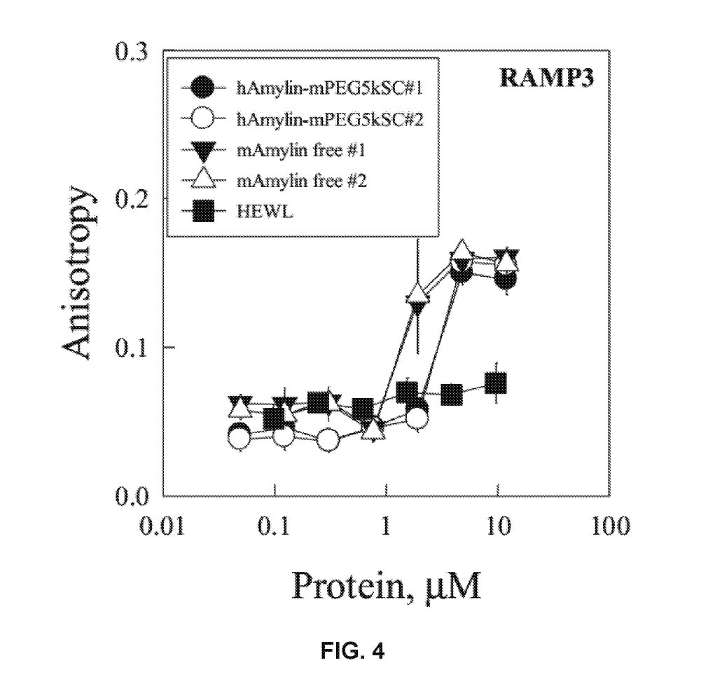

According to FIGS. 3 and 4, binding assay show a similar apparent binding affinity of both free and purified monoPEGylated human amylin for the coreceptors RAMP2 and RAMP3, in duplicate assays (monoPEGylated human amylin C#1,C#2; and free amylin #1 and #2), indicating that the PEG moiety does not interfere with the coreceptor affinity. Control assay using HEWL show non-specific binding to the coreceptor.

### 3—Effect of PEGylation on the Aggregation Profile of Human Amylin.

Purified monoPEGylated human amylin of example 1 was re-suspended in DMSO and diluted to PBS pH 7.4 and allowed to incubate at 37° C. Samples were evaluated for amyloid aggregates by using the amyloid chromogenic probe thioflavin T (ThT).

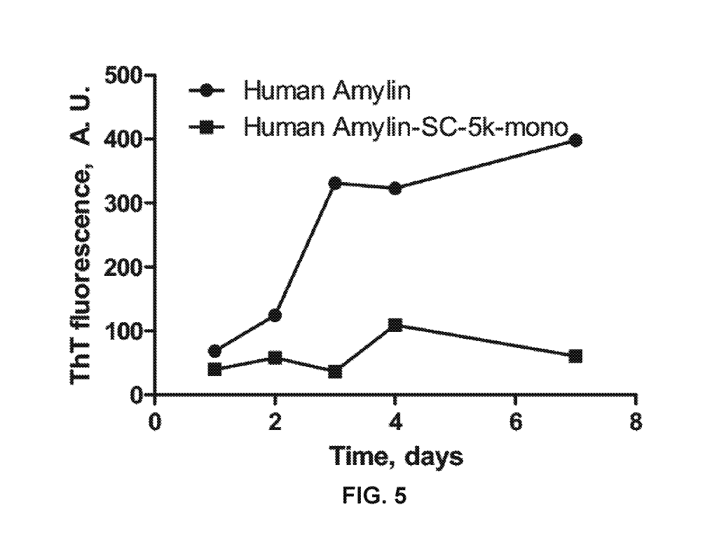

FIG. 5 shows the results. Free Human amylin aggregates very fast. In 3 days, almost all free human amylin was aggregated in amyloid form. MonoPEGylated human amylin, at day 7, still shows no signs of aggregation. This assay proves that the PEGylation process was able to inhibit the agglomeration of human amylin.

4—Pharmacokinetics of the PEGylated Human amylin Product

The pharmacokinetics of the monoPEGylated human amylin product were characterized in vivo in swiss mice. Mice were housed in a temperature-controlled room with a light-dark cycle of 12 h. Water and food were available ad libitum. Two groups were formed, control (free human amylin, not PEGylated) and PEGylated amylin. Animals received 100 uL of saline containing 10 μg of human amylin peptides, either free human amylin or purified monoPEGylated human amylin. Blood was collected retro-orbitally from mice (n=3 mice for each time interval), and the plasma was subjected to ELISA assay for human amylin as provided by the manufacturer (Millipore, Cat Number EZHA-52K—http://www.millipore.com/catalogue/item/ezha-52k).

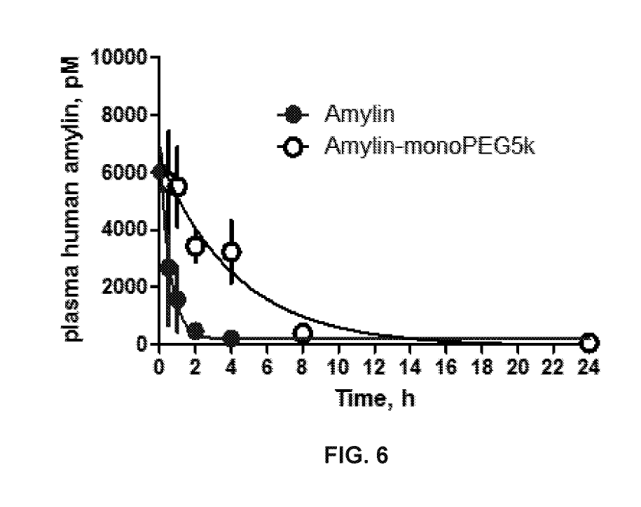

FIG. 6 shows fast plasma decay of free amylin, while the monoPEGylated human amylin shows longer duration of stability in plasma. This assay proves that the process of PEGylation of human amylin is capable of increasing its half-life.

A person skilled in the art may use the teachings of the description and examples given herein to perform the invention in equivalent embodiments which, though not expressly described, perform the same or similar functions to attain the same or similar results, and therefore are encompassed by the scope of protection of the attached claims.

1-25. (canceled)

26. Non-agglomerating bioconjugates of human amylin and polyethyleneglycol, wherein said bioconjugate contains at least one polyethylene glycol unit, covalently bonded to the nitrogen atom forming the alpha and/or epsilon amine moieties (lateral chain) of the lysine 1 residue of the amylin polypeptidic chain, the human amylin sequence being KCNTATCATQRLANFLVHSSNNFGAILSSTNVGSNTY (SEQ ID NO: 1).

27. Non-agglomerating bioconjugates of human amylin and polyethyleneglycol, according to claim 26, having the formula I
(R1-COX)m-R2
where R1 represents a methoxypolyethylene glycol (mPEG) moiety and functional spacers with various average molar masses,
R2 represents human amylin,
X represents NH or O,
m represents the number of units of the mPEG polymer (R1) conjugated to human amylin (R2) obtained from the conjugation of mPEG-succinimidyl with human amylin, including human amylin devoid of Lys1 (des-Lys1), through an amide or ester bond by reaction of primary amine or hydroxyl functional moieties;
or compounds of formula II
(R1X)m-R2
where
R1 represents methoxypolyethylene glycol (mPEG) moiety and functional spacers with various average molar masses,
R2 represents human amylin,
X represents NH or O,
m represents the number of units of the mPEG polymer (R1) conjugated to human amylin (R2) obtained from the conjugation of mPEG-aldehyde with human amylin.

28. Non-agglomerating bioconjugates of human amylin and polyethylene glycol according to claim 26 wherein said human amylin is natural, synthetic or bio-semisynthetic.

29. Non-agglomerating bioconjugates of human amylin and polyethylene glycol according to claim 26 wherein said amylin is in the form of salts, isomers, hydrates, solvates, prodrugs, metabolites, polymorphs or isosters thereof.

30. Non-agglomerating bioconjugates of human amylin and polyethylene glycol according to claim 26 wherein said bioconjugates, as well as pharmaceutical products, medicaments, compositions and associations that comprise said bioconjugates are for use in medical therapy.

31. Non-agglomerating bioconjugates of human amylin and polyethylene glycol according to claim 26 for use in the prevention or treatment of diseases and dysfunctions caused or favored by amyloid deposition or accumulation.

32. Non-agglomerating bioconjugates of human amylin and polyethylene glycol according to claim 31 wherein said diseases or dysfunctions are one or more among hyperglycemia, diabetes, low tolerance to glucose or deficient glucose metabolism, obesity, metabolic syndrome, feeding disorders, atherosclerosis, myocardial infarction, stroke, heart coronary disease, heart diseases in general, Alzheimer disease.

33. Non-agglomerating bioconjugates of human amylin and polyethylene glycol according to claim 31 for use in the prevention or treatment of diabetes mellitus.

34. Non-agglomerating bioconjugates of human amylin and polyethylene glycol according to claim 26 for use in the preparation of low toxicity products useful in the treatment or prevention of diseases caused or favored by amyloid deposition or accumulation.

35. Low toxicity pharmaceutical compositions comprising a therapeutically effective amount of a non-agglomerating bioconjugates of human amylin with polyethylene glycol according to claim 26 and one or more pharmaceutically acceptable excipients.

36. Compositions according to claim 35 additionally comprising one or more active principles distinct from human amylin.

37. Compositions according to claim 36 wherein said one or more active principles distinct from human amylin are chosen from insulin, ions such as zinc or sodium, antidiabetics, antibiotics, antihypertensives, antiretrovirals.

38. Compositions according to claim 37 wherein one said active principle distinct from human amylin is insulin.

39. An adjuvant for the prevention or treatment of diseases comprising a non-agglomerating bioconjugate of human amylin and polyethylene glycol as described in claim 26.

40. Medicament comprising a therapeutically effective amount of a non-agglomerating bioconjugate human amylin and polyethylene glycol as described in claim 26.

41. Method of treatment or prevention of diseases, said diseases caused or favored by amyloid deposition or accumulation comprising the administration to a patient of a therapeutically effective amount of one or more non-agglomerating bioconjugates or human amylin and polyethylene glycol as described in any one of claim 26.

42. Process to obtain a bioconjugate of human amylin and PEG comprising the steps of:
a. Reaction of a human amylin solution with excess mPEG (methoxypolyethylene glycol);
b. interruption of the reaction;
c. purification of the reaction product;
d. drying of the purified product.

43. Process according to claim 42 with steps a and b being:
a. Reaction for 2 h at 25° C. of a 5 mg/mL human amylin solution in the presence of 10 mM PBS (phosphate buffer solution) pH 7.4, and a molar excess of 5 mPEG/1 human amylin;
b. addition of an equal amount of 30% acetonitrile/0.1% trifluoroacetic acid in water to quench the reaction.


SEQUENCE LISTING (1 sequence)

SEQ ID NO: 1 - protein, 37 aa, Artificial Sequence [sequence omitted]